(19) United States
(12) Patent Application Publication
(10) Pub. No.: US 2026/0216494 A1
(43) Pub. Date: Jul. 30, 2026

(54) Catheter Assembly

(71) Applicant: PulseCath B.V., Breukelen (NL)

(72) Inventor: Oren Malchin, Maarssen (NL)

(21) Appl. No.: 19/157,773

(22) PCT Filed: Mar. 14, 2023
(86) PCT No.: PCT/NL2023/050127

§ 371 Date: Aug. 19, 2025

(51) Int. Cl. A61M 60/841; A61M 1/00; A61M 25/01; A61M 25/09; A61M 60/13; A61M 60/139; A61M 60/295; A61M 60/867; A61M 60/894

(52) U.S. Cl. CPC A61M 60/841; A61M 1/87; A61M 60/13; A61M 60/139; A61M 60/295; A61M 60/867; A61M 60/894; A61M 25/0108; A61M 25/09

(57) ABSTRACT

Catheter assembly, including:

a flexible outer tube having a distal aspiration tip;

a flexible inner tube that is insertable into the outer tube, the inner tube having a distal tip configured to protrude from the distal aspiration tip of the outer tube;

the assembly preferably including a guide wire for guiding the outer tube and inner tube into a patient.

### CROSS REFERENCE TO RELATED APPLICATION

The present application is a U.S. National Phase Application pursuant to 35 U.S. C § 371 of International Application No. PCT/NL2023/050127 filed on Mar. 14, 2023, the entire contents of which are herein incorporated by reference as if fully set forth in this description.

### BACKGROUND

The invention relates to a catheter assembly.

Percutaneous Mechanical Circulatory Support (MCS) is increasingly recognized as an important adjunct in the management of challenging coronary anatomies in high risk patients among other conditions.

Heart teams may select patients for Percutaneous Coronary Intervention (PCI) over Coronary Artery Bypass Grafting (CABG) despite the presence of high-risk features like poor V function, high SYNTAX score and hemodynamic instability. Percutaneous MCS may also complement treatment of cardiogenic shock as a rescue or to limit the need for inotropes and vasopressors which are known to increase myocardial oxygen consumption and worsen prognosis.

There is international consensus on the use of percutaneous MCS in cardiovascular care with the purpose to reduce LV (left ventricle) stroke work and myocardial oxygen demand while maintaining systemic and coronary perfusion in the setting of cardiogenic shock or to provide hemodynamic support during complex cardiac procedures including HR-PCI and certain high-risk ablation procedures.

A pulsatile device like an Intra-aortic Balloon Pump (IABP) offers diastolic augmentation to increase myocardial perfusion and reduces afterload during systole to promote forward flow from the LV. However, the hemodynamic support is only very modest. A known prior art catheter system includes a iVAC 2L (https://www.pulsecath.com/ivac-2l/) which is a cardiac support catheter that is inserted across the aortic valve into the LV and that is driven by a genuine IABP (intra-aortic balloon pump) console. It can generate a sufficient, pulsatile flow on top of the existing patient cardiac output.

The iVAC system has shown favorable results. However, because of its diameter, only selected patients with a reasonably well condition and inner size of the artery at the insertion site, are suited to be treated with support of the (17 Fr) iVAC 2L. Insertion using a sheathless technique is not considered feasible.

As follows from the publication “PulseCath iVAC2L: next-generation pulsatile mechanical circulatory support”, Bastos et al., 14 Jan. 2020, Future Cardiology, Future Medicine Ltd., the iVAC2L system leads to good results. Also, it is indicated that a new version of the iVAC system will feature an improved 16F profile to accommodate smaller ilio-femoral anatomies for its insertion and reduce access related complications. This new version would not require a separate (larger) sheath but follows a ‘sheathless’ insertion.

### SUMMARY

The present invention aims to provide an improved catheter assembly and system. In particular, an aspect of the present invention aims to further reduce the risk of vascular complications and achieve an even lower resistance in the LV outflow tract. In particular, the invention aims to provide a (e.g. pulsatile cardiac support) catheter having a relatively small diameter, preferably a diameter of only 16Fr (i.e. 16 French scale), in an economical efficient manner. The resulting catheter can reduce the risk of vascular complications, when used in medical treatment of a patient. Also, an aim is to provide a new catheter device that, in terms of safety, efficacy, usability and applicability, matches or exceeds the very good performance of the prior art catheter system. Moreover, an aim of the invention is to provide a means for applying pulsatile cardiac support safely in case of high risk patients, e.g. in case of insertion via a calcified artery (e.g. a femoral artery).

According to an aspect of the invention, one or more of these goals can be achieved by the features of claim 1.

According to an aspect of the invention there is provided a catheter assembly, including:
a flexible outer tube having a distal aspiration tip;
a flexible inner tube that is insertable into the outer tube, the inner tube having a distal tip configured to protrude from the distal aspiration tip of the outer tube;

the assembly preferably including a guide wire for guiding the outer tube and inner tube into a patient.

It has been found that in this way, an improved catheter assembly and respective system can be provided. In particular, the catheter assembly can be used in a ‘sheathless’ insertion, wherein the protruding tip of the flexible inner tube can provide for safe insertion, even in case of high risk patients.

In particular, the inner tube is only used for insertion and positioning the catheter, for example in the LV. According to an embodiment, an elevated shape of the distal tip of the inner tube can provide the possibility to insert the catheter through the skin, percutaneous approach without using a sheath as introducer. It has been found that this option of not using introducer sheath can decrease the OD (outer diameter) size from 19.5 Fr, to 17 or 16 fr. An advantage is reduction of puncture size, reducing the risk of bleeding, and providing the possibility of using the device in smaller femoral artery ID (inner diameter) size, which significantly increases the potential number of patients that can be treated. Further, according to an embodiment, a resulting change in the tip of the catheter (i.e. bigger hole c.q. increase of blood passage dimension) increases a surface of inlet hole size, which will reduce resistance during operation (i.e. in case of a vacuum, or under-pressure, in particular during the pumping out of blood from the LV). This can positively affect the efficacy of pumping and can lead to increase of volume and higher performance of the pump. This increases cardiac output and mean pressure.

Further extra advantageous embodiments of the invention are provided in the dependent claims.

### BRIEF DESCRIPTION OF THE FIGURES

Non-limiting examples of the invention will now be explained in more detail with reference to the drawing. Therein shows:

FIG. 1 a side view of a prior art catheter assembly;

FIG. 2 a pump of the assembly of FIG. 1;

FIG. 3 schematically a side view of components of a non-limiting embodiment of the present invention;

FIG. 4 schematically part of the embodiment of FIG. 3 after assembly, in an opened view;

FIG. 5 shows an aspiration tip of the outer tube of the embodiment of FIG. 3, in perspective view;

FIG. 6 shows a longitudinal cross-section of the aspiration tip depicted in FIG. 5; and

FIG. 7 shows a longitudinal cross-section of a detail of an example of the inner tube of the embodiment of FIG. 3.

In the present application, corresponding or similar features are denoted by corresponding or similar reference signs.

### DETAILED DESCRIPTION

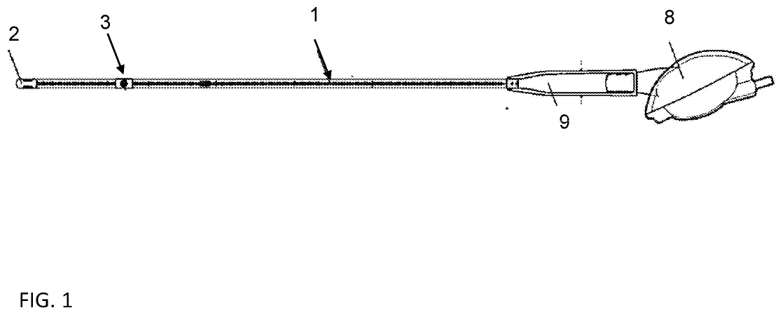

FIG. 1 depicts a known catheter assembly (see Bastos et al.), including a flexible tube 1 (lumen) having a distal aspiration tip 2. A diameter of the known tube is 17 Fr. The tube 1 includes a bidirectional valve 3 at a relatively short distance (in particular in the range of 6-8 cm, e.g. 73 mm) from a distal end of the aspiration tip 2. The valve 3 has a lateral blood outlet port for discharging blood during a diastolic pump phase (see below). The valve 3 closes the outlet port during a systolic pump phase.

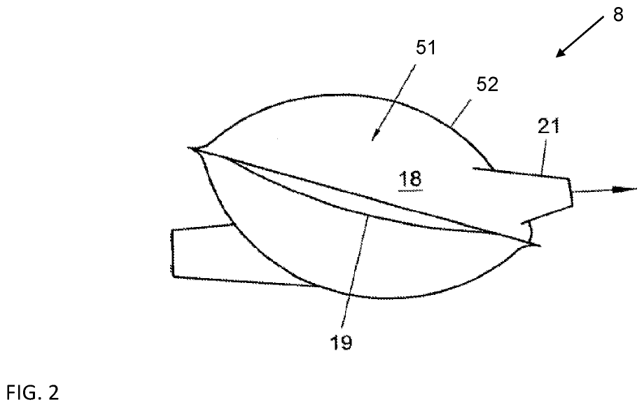

A pump 8 is connected to a proximal end of the flexible tube 1 (via a suitable connector 9). A non-limiting example of the pump 8 is shown in FIG. 2 (in partly opened view). It is preferred that the pump 8 (e.g. a catheter pump 8) has a relatively simple configuration, providing reliable and durable blood flow action. To that aim the pump 8 can include a displacement structure 51 for, while in operation, alternatingly applying suction and pressure, in particular for cyclically driving fluid displacement through the flexible tube 1, wherein a flow path between the displacement structure and the tube 1 is preferably unobstructed (i.e. continuously open) after assembly. The displacement structure 51 of the pump 8 preferably includes a (flexible) membrane 19.

For example, the pulsatile pump 8 can have a displacement structure in the form of a rigid housing 52 enclosing a chamber 18 divided by a flexible membrane 19. The pump 8 can be connected directly to the flexible tube 1 (or indirectly via the intermediate connector 9), the tube 1 projecting from that displacement structure, wherein the tube 1 bounds an internal flow channel for leading fluid (i.e. blood, during operation) to and from the downstream valve 3 of the tube 1. The tube's flow channel can communicate with a portion of the chamber 18 on a distal side of the membrane 19. A portion of the chamber 18 on a proximal side of the membrane 19 can communicate with a sleeve 21 for connection to a pneumatic conduit communicating with a pneumatic drive system (known per se). For example, the system can include a driver configured to be synchronized with a heartbeat of the patient, as will be appreciated by the skilled person.

For example, the displacement structure can be driven via the pneumatic conduit for alternatingly applying suction for displacing fluid (i.e. blood) from the tube's aspiration tip towards the valve 3 (diastolic phase) and for applying pressure for displacing fluid (i.e. blood) from the displacement structure 51 into the tube 1 (systolic phase), thus driving a flow through the tube 1 which reverses in a pulsating manner.

The known catheter is usually inserted into a patient using a 18 Fr. sheath (not shown) and a guide wire.

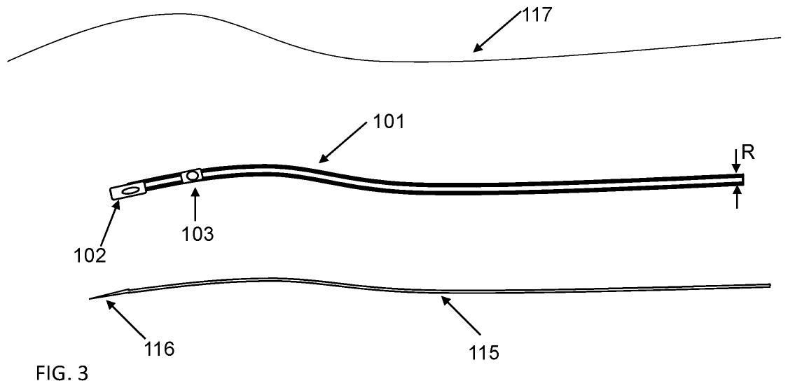

FIG. 3 shows and improved catheter assembly (in disassembled state), that can be used with great advantage in case of high risk patients.

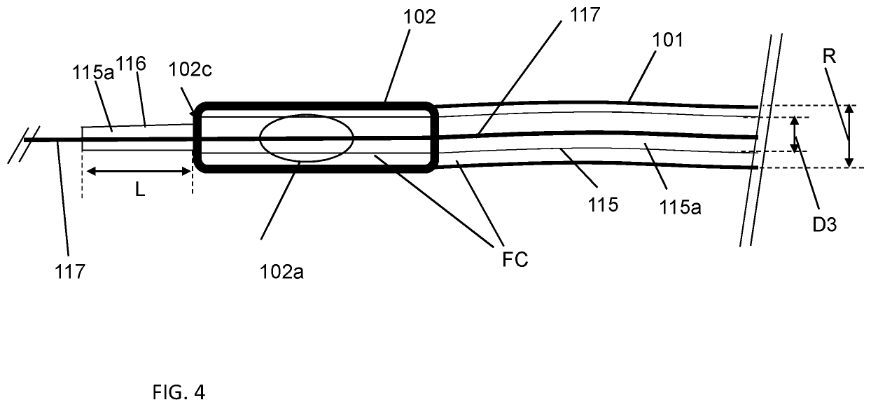

The improved catheter assembly includes:
a flexible outer tube 101 having a distal aspiration tip 102;
a flexible inner tube 115 that is insertable into the outer tube 101 (via a proximal end of the inner tube), the inner tube having a distal tip 116 configured to protrude from the distal aspiration tip 102 of the outer tube 101 (the distal tip 116 protruding from the distal aspiration tip 102 after assembly, see FIG. 4).

The assembly preferably includes a (flexible) guide wire 117 for (slidably) guiding the outer tube 101 and inner tube 115 into a patient, the inner tube 115 in particular being configured to (slidably) receive the guide wire 117 (to be guided by the guide wire 117). Guide wires 117 as such are commonly known; the length of the guide wire 117 is longer than the length of the outer tube 101 (and a length of the inner tube) that is to be guided into the patient. The diameter of the guide wire 117 is smaller than an inner diameter the outer tube 101, and smaller than an inner diameter of the inner tube 115. The guide wire 117 can be a solid metal or steel wire, or a compound wire e.g. having a core embedded in one or more layers, or the-like.

FIG. 4 shows an assembled state of the catheter system, wherein a distal end of the outer tube 101 has (relatively concentrically) received the inner tube 115, both tubes 101, 115 being (slidably) guided by a respective guide wire 117 (extending through the inner tube 115). It is preferred that the inner tube 115 is arranged inside the outer tube 101, such that the distal section 116 of inner tube 115 axially protrudes out of a distal opening 102c of the aspiration tip 102 of the outer tube 101 (as in FIG. 4), during the receiving of and guiding by the guide wire 117 (for guiding the aspiration tip 102 towards and into the heart of a patient). After assembly, the inner tube 115 is releasably held in the outer tube 101 (so that the inner tube 115 can be removed/withdrawn from the outer tube 101, via a proximal end of the outer tube 101, once the outer tube 101 has been positioned correctly in a patient).

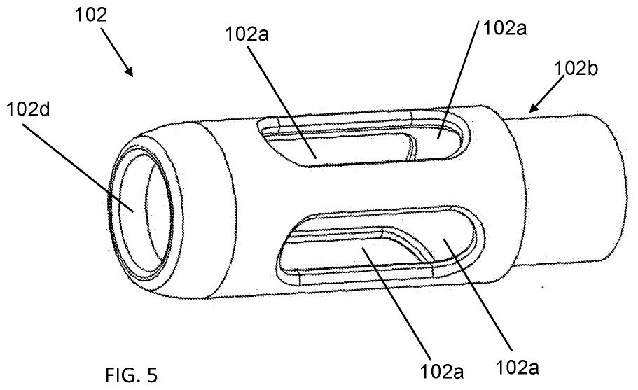

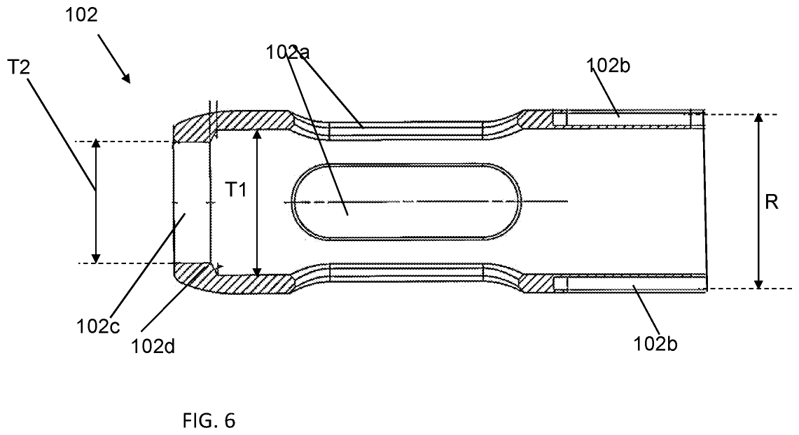

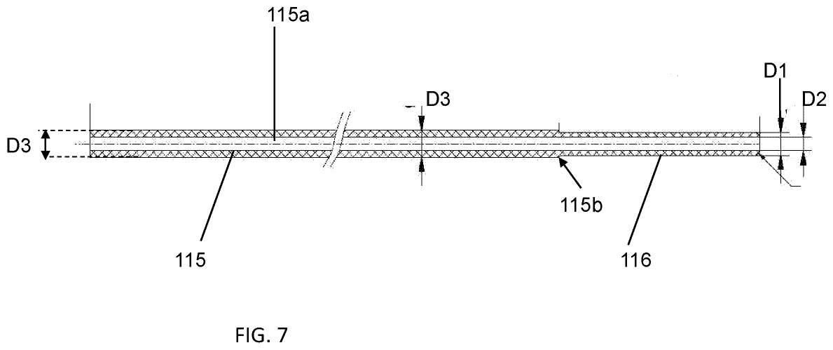

FIGS. 5 and 6 show an example of the aspiration tip 102 of the outer tube 101. A distal part of an example of the inner tube 115 is shown in FIG. 7.

The outer tube 101 can generally have the same configuration as the known tube 1 (depicted in FIG. 1), but preferably has a maximum outer diameter R that is smaller than 6 mm, for example an outer diameter R of 16 Fr. In a non-limiting example, the outer tube 101 can be substantially made of PTFE (polytetrafluorethyleen).

The outer tube 101 is connectable to a blood pump 8 (known as such, see above), in particular a pump 8 including a displacement structure 51 (e.g. membrane 19) for, while in operation, alternatingly applying suction and pressure, in particular for cyclically driving fluid displacement through said outer tube 101. Also, it is preferred that the outer tube 101 has an integrated valve 103, located near the aspiration tip 102 (e.g. at a distance in the range of 6-8 cm, e.g. 73 mm from a distal end of the tip 102). The respective valve section of the outer tube 101 can include a blood discharge port at the integrated valve 103, for discharging blood during a diastolic pump phase (see above), wherein the valve 103 is configured to close the discharge port during a systolic pump phase (allowing blood to pass the valve 103 from the pump 8 to the aspiration tip 102). The outer tube 101 can provide an integrated flow path between the displacement structure and the integrated valve that is unobstructed.

An example of the integrated aspiration tip 102 of the flexible outer tube 101 is shown in FIGS. 5 and 6. For example, the integrated tip 102 can be made of metal (e.g. titanium or stainless steel). The tip 102 can be a substantially cylindrical element (wall), having a number of side ports 102a allowing fluid communication through the wall of the tip 102. The integrated distal aspiration tip 102 of the outer tube 101 can have a maximum outer diameter R that is smaller than 6 mm, for example an outer diameter R of 16 Fr. An inner diameter T1 of the tip 102 can be about 1 mm smaller than the tip's outer diameter R, for example an inner diameter T1 in the range of about 4-5 mm (for example about 4.7 mm).

The aspiration tip 102 can be mounted onto an axial end of the outer tube 101 in various ways, e.g. using suitable adhesive and/or a suitable welding process. In this example, the tip 102 has a proximal groove 102b for receiving an opposite end of the outer tube 101 (the tube 101 being attached to the tip 102 via that groove 102b after assembly). A distal opening 102c of the present aspiration tip 102 is open, allowing axial passage of the distal tip 116 of the inner tube 115. In the present example, the distal axial opening 102c of the aspiration tip 102 is defined by an inner flange (circular edge) 102d, the resulting opening 102c having a circular contour/shape with a diameter T2 that is smaller than the inner diameter T1 of a main section of the tip 102. The difference between the inner diameter T1 of the tip and the diameter T2 of the distal opening 102c can be relatively small, e.g. in the range of 0.1-2 mm, preferably a difference in the range of 0.4-1.2 mm (for example a difference of about 0.8 mm).

The inner tube 115 of the assembly can be configured in various ways. The tip 116 of the inner tube 115 can e.g. be a tapered tip, and/or a tip having a smaller outer diameter than a diameter of a proximal section of the inner tube 115. The inner tube 115 can be made of various materials, preferably PTFE. In addition, the inner tube can include one or more (marker) materials that a radio-opaque (e.g. barium sulfate), for tracing/visibility using e.g. x-rays.

According to an embodiment, the inner tube 115 can have a length of e.g. at least 100 cm, for example, a length in the range of 110-120 cm (for example a range of about 110-112 cm). It is preferred that the inner tube 115 is longer than the respective outer tube 101, such that an inner tube's proximal end protrudes from a proximal end of the outer tube after assembly.

The inner tube 115 can e.g. consist of a proximal circle-cylindrical tube section (that extends via the aspiration tip 102 towards the valve section of the outer tube 101) and an integrated (e.g. made in one piece) distal circle-cylindrical tip 116 that extends away from the outer tube 101 via the distal opening 102c of the aspiration tip 102. In the present example, the inner tube 115 is of relatively simple construction, including only two sections of different diameters. It will be appreciated that the inner tube 115 can also include more than two sections that have mutually different diameters.

In particular, the inner tube 115 defines a central passage 115a (extending between opposite open ends of the inner tube 115) for receiving the guidewire 117 (so that the inner tube 115 can be guided by the guidewire 117 during entry into a patient). As will be appreciated, an inner diameter D2 of the inner tube 115 is preferably larger than an outer diameter of the guidewire 117. For example, the inner diameter D2 of the inner tube can be 3 mm or smaller (for example a diameter D2 in the range of 1-3 mm, e.g. 2 mm. Also, the inner tube 115 preferably has opposite open ends (one of the open ends being a distal end of the tube's tip 116) for entry and exit of the guide wire 117.

It is preferred that an outer side of the inner tube 115 sealingly engages the distal axial opening 102c of the aspiration tip 102, after assembly (to locally prevent blood flow between opposite sides or edges of the tip 102 and the inner tube 115).

In an example, as shown in FIG. 7, a proximal section of the inner tube 115 can have an outer diameter D3 that is smaller than the inner diameter of the respective outer tube 101 and smaller than the inner diameter T1 of the aspiration tip 102 of the outer tube 101. In order to achieve stable axial positioning, preferably, the outer diameter D3 of the proximal section of the inner tube 115 is larger than the diameter T2 of the exit opening 102c of the aspiration tip 102. In this way, the inner flange (circular edge) 102d of the aspiration tip 102 can act as an end stop for positioning the inner tube 115 with respect to the outer tube 101, in particular for mechanically contacting (and preferably fluidly sealing with) an outer rim 115b of the inner tube 115. In this example, said outer rim 115b is provided by a distal edge of the proximal section of the inner tube 115 (i.e. it is located at the proximal start of the axially protruding distal section (tip) 116 of that tube 115).

In case the proximal section of the inner tube 115 has an outer diameter D3 that is smaller than the inner diameter of the respective outer tube 101, the opposite sides of the two tubes 101, 115 can define an intermediate blood flow passage, allowing blood to flow from the distal aspiration openings (side ports) 102a of the outer tube's tip 102 to respective proximal ends of the two tubes (which can be used for blood pressure detection during catheter insertion).

The outer diameter D1 of the distal tip 116 of the inner tube 115 is preferably smaller than the outer diameter D3 of the proximal section of that tube 115, and preferably the same as or smaller than the diameter T2 of the distal opening 102c of the aspiration tip 102, so that the distal tip 116 of the inner tube 115 can pass the distal opening 102c of the aspiration tip 102 towards its operating position (shown in FIG. 7), at least during assembly.

In the example shown in FIG. 7, the distal tip 116 of the inner tube 115 has a substantially constant outer diameter D3 (alternatively, e.g. a varying outer diameter can be provided, such as a tapered or frusco-conical tip 116). For example, a maximum outer diameter D1 of at least a butt end of the tip 116 of the inner tube 115 (the butt end being located axially from the distal end of the aspiration tip 102 of the outer tube 101 after assembly) can be e.g. 4 mm, for example an outer diameter D1 in the range of 3.9-3.1 mm, such as an outer diameter D1 of 3.4 mm.

After assembly, the inner tube's tip 116 preferably protrudes out of the flexible outer tube 101 over a distance L of at least 2 cm from the aspiration tip of the outer tube (i.e. the distance L being measured in axial direction between the butt ends of the two tips 102, 116, see FIG. 4), preferably a distance L in the range of 2-5 cm, more preferably distance L in the range of 2-4 cm, for example a distance L of about 3 cm.

The resulting protruding tip 116 of the flexible inner tube 115 can provide significant advantages over known percutaneous mechanical circulatory support catheter systems. As is mentioned before, the resulting catheter assembly can be used in a ‘sheathless’ insertion, wherein the protruding tip of the flexible inner tube can provide for safe insertion, even in case of high risk patients.

During insertion into a patient's body, an operator can hold a proximal part of the catheter assembly and prevent displacement of the inner tube 115 inside the outer tube 101 until final positioning (of the tip 102) in a heart's LV. Insertion generally uses the guidewire 117 (being located in the inner tube, see FIG. 4). Next, the operator can remove (withdraw) the inner tube 115 out from outer tube 101 (as well as the guidewire 117) while keeping the distal tip of the outer tube in the LV. After full removal of the inner tube (and guidewire), the outer tube can be connected to the membrane pump 8. Then, the apertures in the distal tip 102 of the outer tube 101, including the distal opening 102c that was initially used by the inner tube during assembly placement, can provide aspiration access for entry of blood into the outer tube 101 (due to pump action).

It follows that the inner tube 115 provides improved insertion and positioning of the outer tube 101, after which the inner tube is removed from the outer tube 101.

According to an embodiment (as mentioned above), a difference of an outer diameter size of the inner tube 115 and an inner diameter size of the outer tube 101 allows blood flow during operation via a blood flow passage defined between the two tubes 101, 115 (the blood flow passage extending from the side ports 102a of the aspiration tip 102 to proximal tube ends). Also, a second blood flow passage is available (extending between opposite ends of the inner tube 115), provided by the interior of the inner tube 115 as such. The two blood flow passages are separated by the inner tube 115 (after assembly), and can be used for example for measuring respective blood pressures of blood flowing from those passages to an assemblies proximal portion that is located outside the patient (using commonly known blood pressure measuring equipment). Blood pressure monitoring via the catheter assembly can be carried out during catheter assembly insertion, for example to verify if the distal tip of the inner tube 115 has crossed an aortic valve. The pressure in the heart's LV is usually different from aortic pressure, therefore, when the distal tip of the inner tube 115 has passed the aortic valve, and the aspiration tip 102 of the outer tube 101 has not passed that valve, a resulting blood pressure difference (of blood emanating at the proximal ends of the two tubes) can be detected (at the proximal ends of the tubes) so that the operator knows that the tip is in the LV (e.g. without having to rely on x-ray monitoring).

While this disclosure includes specific example embodiments, it will be apparent to one of ordinary skill in the art that various changes in form and details may be made in these example embodiments without departing from the scope of the claims. The example embodiments described herein are to be considered in a descriptive sense only, and not for purposes of limitation.

For example, the outer tube can be called catheter, and can optionally be defined as an elongated tubular (flexible) medical device that is to be used for Percutaneous Coronary Intervention, for example in cooperation with a pulsatile device like an Intra-aortic Balloon Pump (IABP). The outer tube can in particular consists of a circle-cylindrical wall, concentrically enclosing a single fluid channel (e.g. a single lumen for guiding blood or another fluid).

1. A catheter assembly, including:
a flexible outer tube having a distal aspiration tip; and
a flexible inner tube that is insertable into the flexible outer tube, the flexible inner tube having a distal tip configured to protrude from the distal aspiration tip of the flexible outer tube.

2. The catheter assembly of claim 1, wherein a maximum outer diameter of a butt end of the distal tip of the flexible inner tube is 4 mm.

3. The catheter assembly of claim 1, wherein a length of the flexible inner tube is at least 100 cm.

4. The catheter assembly of claim 1, wherein the distal tip of the flexible inner tube protrudes over a distance of at least 2 cm from the distal aspiration tip of the flexible outer tube.

5. The catheter assembly of claim 1, wherein a proximal section of the flexible outer tube includes a blood discharge port.

6. The catheter assembly of claim 5, wherein the flexible outer tube is connectable to a blood pump including a displacement structure for, while in operation, alternatingly applying suction and pressure, for cyclically driving fluid displacement through the flexible outer tube.

7. The catheter assembly of claim 6, wherein the flexible outer tube includes an integrated a flow path between the displacement structure and an integrated valve included in the flexible outer tube is unobstructed.

8. The catheter assembly of claim 1, wherein the flexible inner tube is releasably coupled to the flexible outer tube.

9. The catheter assembly of claim 1, wherein the flexible inner tube is made of PTFT.

10. The catheter assembly of claim 1, wherein an outer side of the flexible inner tube sealingly engages a distal opening of the distal aspiration tip of the flexible outer tube.

11. The catheter assembly of claim 1, wherein a proximal section of the flexible inner tube has an outer diameter that is smaller than an inner diameter of the flexible outer tube, such that opposite sides of the flexible inner tube and the flexible outer tube define an intermediate blood flow passage.

12. The catheter assembly of claim 11, wherein the flexible inner tube defines a second blood flow passage that is separate from the intermediate blood flow passage.

13. A heart assist catheter pump system, including:
a catheter comprising: (i) a flexible outer tube having a distal aspiration tip, and (ii) a flexible inner tube that is insertable into the flexible outer tube, the flexible inner tube having a distal tip configured to protrude from the distal aspiration tip of the flexible outer tube; and
a pump having a displacement structure for, while in operation, alternatingly applying suction and pressure, for cyclically driving fluid displacement through the flexible outer tube of the catheter.

14. The catheter assembly of claim 2, wherein the maximum outer diameter of the butt end of the distal tip of the flexible inner tube is in a range of 3.9-3.1 mm.

15. The catheter assembly of claim 2, wherein the maximum outer diameter of the butt end of the distal tip of the flexible inner tube is 3.4 mm.

16. The catheter assembly of claim 1, further comprising:
a guide wire for guiding the flexible outer tube and the flexible inner tube into a patient.

17. The heat assist catheter pump system of claim 13, wherein the catheter further comprises:
a guide wire for guiding the flexible outer tube and the flexible inner tube into a patient.

18. The catheter assembly of claim 5, wherein the proximal section of the flexible outer tube further includes an integrated valve.

19. The catheter assembly of claim 7, wherein the displacement structure comprises a membrane.

20. The catheter assembly of claim 1, wherein the flexible inner tube includes a radio-opaque material.
